(19) United States
(12) Patent Application Publication
(10) Pub. No.: US 2010/0286587 A1
(43) Pub. Date: Nov. 11, 2010

(54) SUBLINGUAL ELECTRICAL DRUG DELIVERY

(76) Inventor: Yossi Gross, Moshav Mazor (IL)

Correspondence Address: SUGHRUE MION, PLLC, 2100 PENNSYLVANIA AVENUE, N.W., SUITE 800, WASHINGTON, DC, 20037, US

(21) Appl. No.: 12/437,149

(22) Filed: May 7, 2009

(51) Int. Cl. A61N 1/30

(52) U.S. Cl. 604/20

(57) ABSTRACT

Apparatus is provided that includes an oral housing, which is configured to be placed at least partially in a mouth of a subject, and which comprises a drug, and two or more electrodes. The apparatus further includes a power supply, and circuitry coupled to the power supply and the electrodes, and configured to drive the electrodes to drive, into oral tissue of the subject, a current having a frequency of between 30 and 300 kHz. Other embodiments are also described.

### FIELD OF THE INVENTION

The present invention relates generally to drug delivery apparatus and methods, and specifically to techniques for electrical enhancement of drug delivery.

### BACKGROUND OF THE INVENTION

In sublingual drug administration, a drug is absorbed directly into the bloodstream through tissue under the tongue. Sublingual administration generally is quicker and more efficient than oral administration, because passage of the drug through the digestive track is avoided.

Iontophoresis electrically induces transport of charged ions by applying low-level, direct current (DC) to a solution of the drug. Since like electrical charges repel, the application of a positive current drives positively charged drug molecules away from the electrode and into the tissues; similarly, a negative current drives negatively charge ions into the tissues.

US Patent Application Publication 2004/0158194 to Wolff et al., which is incorporated herein by reference, describes drug dosage forms that are housed in oral devices, and methods for controlled drug release. The oral devices are permanently or removably inserted in the oral cavity and refilled or replaced as needed. The controlled drug release may be passive, based on the dosage form, or electronically controlled, for a high-precision, intelligent, drug delivery. Additionally, the controlled release may be any one of the following: release in accordance with a preprogrammed schedule, release at a controlled rate, delayed release, pulsatile release, chronotherapeutic release, closed-loop release, responsive to a sensor's input, release on demand from a personal extracorporeal system, release in accordance with a schedule specified by a personal extracorporeal system, release on demand from a monitoring center, via a personal extracorporeal system, and release in accordance with a schedule specified by a monitoring center, via a personal extracorporeal system. Drug absorption in the oral cavity may be assisted by an electrotransport mechanism. The oral devices require refilling or replacement at relatively long intervals of weeks or months, maintain a desired dosage level in the oral cavity, hence in the gastrointestinal tract, for extended periods, address situations of narrow drug therapeutic indices, and by being automatic, ensure adherence to a prescribed medication regimen.

US Patent Application Publication 2007/0191757 to Steiner et al., which is incorporated herein by reference, describes methods, devices and kits for sublingual drug delivery using iontophoresis. An active agent can be administered sublingually by placing a solid oral dosage form containing the active agent in the sublingual region of a patient and applying iontophoresis for a suitable period of time. Preferably up to 4 mA of current are applied to the sublingual region. Different time ranges can be used to administer iontophoresis; preferably iontophoresis is administered for up to two minutes at a time. Any suitable device for administering iontophoresis to the sublingual region may be used. The preferred device is a hand-held device that contains a handle, two electrodes, one of which is located on the handle and the other of which is attached to the end of the handle, and a connection to a power source. Optionally, the device contains a timer, which can be used turn off the current at a preset time. The device can be used to administer an active agent by iontophoresis to the sublingual region of a patient, by attaching the second electrode of the device to a solid oral dosage form containing the active agent to be administered. A kit contains the device for administering iontophoresis and one or more solid oral dosage forms, preferably in the form of one or more tabs or wafers. The tabs or wafers may be completely dissolvable or edible, or may contain a non-edible and non-dissolvable component. In a preferred embodiment, the solid oral dosage form contains insulin or an analog thereof and one or more excipients, preferably EDTA and citric acid.

US Patent Application Publication 2003/0191426 to Lerner et al., which is incorporated herein by reference, describes a device to enhance the delivery of a drug or other substance of interest into a selected organ or tissue, comprising special electrodes, one of the electrodes carrying a container with the selected drug or other substance of interest, the electrodes being capable of being positioned at preselected locations of the organ or tissue. The electrodes are all connected with a selected energy source which generates and maintains an energy field before and during the enhanced delivery of the substance, under the influence of which delivery is accomplished in a direction from the active to the passive electrode and into the organ or tissue. The energy source may be selected from suitable sources providing an electric field, a magnetic field, ultrasonic waves, high energy waves like laser beams, or a combination thereof. Further a method for the enhanced delivery of the drug or other substance of interest to an internal organ or target tissue of an organism, for example the brain, bypassing the blood-brain barrier, is described.

U.S. Pat. No. 5,298,017 to Theeuwes et al., which is incorporated herein by reference, describes an iontophoretic agent delivery device, having a layered structure and peripheral insulation, wherein ion transport occurs through two opposing surfaces of the device. The device is especially suited to agent delivery through body surfaces exposed to body fluids. A method of delivering an agent through a body surface exposed to body fluids is also described.

PCT Publication WO 01/13989 to Sun et al., which is incorporated herein by reference, describes techniques for transporting a molecule through a mammalian barrier membrane of at least one layer of cells. The techniques include ablating said membrane with an electric current from a treatment electrode, and utilizing a driving force to move said molecule through said perforated membrane.

The following patents and patent application publications, all of which are incorporated herein by reference, may be of interest:

U.S. Pat. No. 4,020,558 to Cournut et al.

U.S. Pat. No. 4,959,052 to Cox

U.S. Pat. No. 5,090,903 to Taylor et al.

U.S. Pat. No. 5,190,053 to Meer

U.S. Pat. No. 5,196,002 to Hanover et al.

U.S. Pat. No. 5,584,688 to Sakuma et al.

U.S. Pat. No. 6,006,130 to Higo et al.

U.S. Pat. No. 6,148,232 to Avrahami

U.S. Pat. No. 6,533,579 to Komiyama

U.S. Pat. No. 6,618,627 to Lattner et al.

U.S. Pat. No. 6,692,456 to Eppstein et al.

U.S. Pat. No. 6,955,538 to Borch et al.

U.S. Pat. No. 7,172,594 to Biscup

US Patent Application Publication 2004/0147906 to Voyiazis et al.

US Patent Application Publication 2005/0155601 to Steiner et al.

US Patent Application Publication 2007/0202057 to Fankhauser et al.

US Patent Application Publication 2008/0299079 to Meezan et al.

### SUMMARY OF THE INVENTION

In some embodiments of the present invention, a sublingual drug delivery device comprises a drug, two or more electrodes, a power supply, and circuitry configured to drive a low voltage radiofrequency (RF) current between the electrodes, typically having a frequency of between 30 and 300 kHz. The sublingual drug delivery device is configured to be placed at least partially under the tongue of a subject. The RF current generally: (a) dilates blood vessels in the sublingual region, thereby increasing blood flow, (b) increases pore size in the blood vessels, and/or (c) dilates junctions between epithelial cells. As a result, the RF current enhances absorption of the drug into the systemic blood circulation.

The RF current drug delivery techniques of these embodiments of the present invention enhance the delivery of both water-soluble and non-water-soluble drugs. In contrast, iontophoresis generally enhances the delivery of only water-soluble drugs. For some applications, the RF current drug delivery techniques described herein are used in combination with iontophoresis. For other applications, iontophoresis is performed in the absence of the RF current delivery techniques described herein.

There is therefore provided, in accordance with an embodiment of the present invention, apparatus including:

an oral housing, which is configured to be placed at least partially in a mouth of a subject, and which includes a drug, and two or more electrodes;

a power supply; and

circuitry coupled to the power supply and the electrodes, and configured to drive the electrodes to drive, into oral tissue of the subject, a current having a frequency of between 30 and 300 kHz.

For some applications, the circuitry is configured to drive the electrodes to drive the current as an alternating current between the electrodes.

For some applications, the oral housing has a total volume of between 2 and 8 cm3.

For some applications, the oral housing includes L-arginine.

For some applications, the circuitry is configured to drive the electrodes to drive the current as an alternating current serving as a carrier signal, and to apply a lower frequency signal to the carrier signal using amplitude modulation. For example, the lower frequency signal may have a frequency of between 3 and 40 Hz.

For some applications, the oral housing includes the circuitry. Alternatively or additionally, the oral housing includes the power supply.

For some applications, the oral housing includes an anesthetic. For some applications, the circuitry is configured to: during a first period, drive the electrodes to drive the current at a first strength sufficient to facilitate passage of some of the anesthetic into the oral tissue, and during a second period that commences after a conclusion of the first period, drive the electrodes to drive the current at a second strength that is greater than the first strength, and sufficient to enhance passage of the drug into the oral tissue. For some applications, the anesthetic is applied to the oral housing as a layer, and the oral housing further includes a chemical enhancer that at least partially covers the layer of anesthetic.

In an embodiment, the oral housing is configured to position the electrodes such that the circuitry drives the current into buccal tissue of the subject.

Alternatively or additionally, the oral housing is configured to position the electrodes such that the circuitry drives the current into lip tissue of the subject.

In an embodiment, the oral housing includes a sublingual housing, which is configured to be placed at least partially under a tongue of the subject. For some applications, the sublingual housing is U-shaped.

There is further provided, in accordance with an embodiment of the present invention, a method for delivering a drug to a subject, including:

receiving a oral drug delivery device including a drug, two or more electrodes, a power supply, and circuitry coupled to the power supply and the electrodes, and configured to drive the electrodes to drive, into oral tissue of the subject, a current having a frequency of between 30 and 300 kHz; and

placing the drug delivery device at least partially in a mouth of the subject.

In an embodiment, placing includes placing the drug delivery device at least partially under a tongue of the subject.

For some applications, placing includes placing the drug delivery device at least partially in the mouth such that at least some of the electrodes are positioned such that the circuitry drives the current into buccal tissue of the subject. Alternatively or additionally, placing includes placing the drug delivery at least partially in the mouth such that at least some of the electrodes are positioned such that the circuitry drives the current into lip tissue of the subject.

For some applications, receiving includes receiving the oral drug delivery device including the circuitry, the circuitry being configured to drive the electrodes to drive the current as an alternating current between the electrodes.

For some applications, receiving includes receiving the oral drug delivery device, the device including L-arginine.

For some applications, receiving includes receiving the oral drug delivery device including the circuitry, the circuitry configured to drive the electrodes to drive the current as an alternating current serving as a carrier signal, and to apply a lower frequency signal to the carrier signal using amplitude modulation.

For some applications, receiving includes receiving the oral drug delivery device, the device including an anesthetic. For example, receiving may include receiving the oral drug delivery device that includes the anesthetic, the anesthetic being applied thereto as a layer, the device further including a chemical enhancer that at least partially covers the layer of anesthetic.

There is still further provided, in accordance with an embodiment of the present invention, a method for delivering a drug to a subject, including:

placing the drug in a mouth of the subject;

placing two or more electrodes in the mouth; and

driving a current between the electrodes having a frequency of between 30 and 300 kHz.

In an embodiment, placing the drug and the electrodes includes placing the drug and the electrodes at least partially under a tongue of the subject.

For some applications, driving includes driving the current as an alternating current between the electrodes. For some applications, driving includes driving the current as an alternating current serving as a carrier signal, and applying a lower frequency signal to the carrier signal using amplitude modulation.

The present invention will be more fully understood from the following detailed description of embodiments thereof, taken together with the drawings, in which:

### BRIEF DESCRIPTION OF THE DRAWINGS

FIGS. 1A and 1B are schematic side-view and cross-sectional illustrations, respectively, of a sublingual drug delivery device, in accordance with an embodiment of the present invention.

### DETAILED DESCRIPTION OF EMBODIMENTS

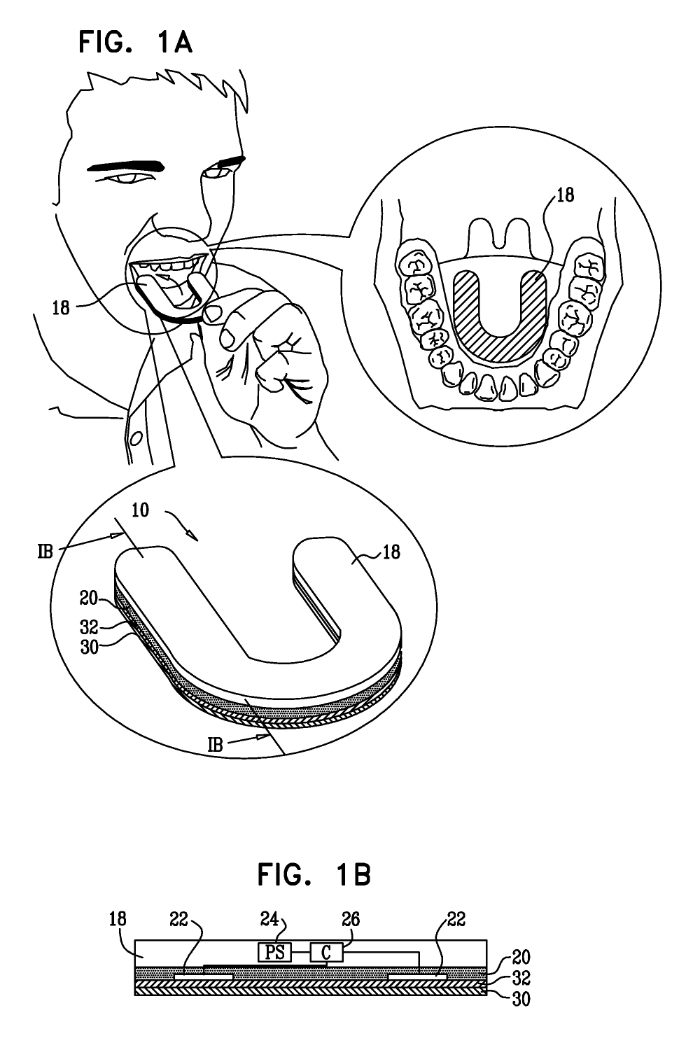

FIGS. 1A and 1B are schematic side-view and cross-sectional illustrations, respectively, of a sublingual drug delivery device 10, in accordance with an embodiment of the present invention. Sublingual drug delivery device 10 is configured to be placed at least partially under a tongue of a subject, and may be U-shaped, for example, as shown in FIG. 1A. Device 10 typically comprises a plurality of layers, which comprise respective components of the device, as described hereinbelow. Typically, device 10 has a total volume of between about 2 and about 8 cm3, such as between about 2 and 6 cm3, which allows the device to be placed under the tongue.

Device 10 comprises a housing 18, which comprises or is coupled to a drug 20. For example, one of the layers of the device may comprise a coating of the drug. For some applications, a portion of the housing is shaped so as to define a handle, which is configured to extend out of the mouth. The handle allows the subject or somebody else to conveniently insert the device under the tongue and subsequently remove it.

Housing 18 further comprises two or more electrodes 22. At least one of the electrodes is positioned in a vicinity of and/or in contact with the drug. For example, the drug may be positioned between the electrodes. For some applications, housing 18 comprises a hydrophobic portion between the electrodes, which enhances current application into the tissue adjacent to the electrodes by reducing current flow adjacent to the hydrophobic portion, directly between the electrodes. The electrodes typically comprise a conductive metal, and may be shaped as a foil or screen. For some applications, one or more of the electrodes are positioned on a surface of housing 18 that faces the floor of the mouth when the housing is placed under the tongue. Alternatively or additionally, one or more of the electrodes are positioned on a surface of the housing that faces the undersurface of the tongue when the housing is placed under the tongue.

For some applications, at least a first one of the electrodes is positioned on a first surface of the housing that faces the floor of the mouth, and at least a second one of the electrodes is positioned on a second surface of the housing opposite to the first surface, which second surface faces the undersurface of the tongue when the housing is placed sublingually. Because electrodes are positioned on opposite sides of the housing, at least a portion of the current travels between the electrodes, through tissue of the floor of the mouth and/or the undersurface of the tongue.

Device 10 further comprises a power supply 24, and circuitry 26 coupled to the power supply and the electrodes. For some applications, housing 18 comprises power supply 24, as shown in FIG. 1B, in which case the power supply typically comprises a battery. Alternatively, the power supply is configured to remain outside of the mouth of the subject, in which case the power supply is typically coupled to the housing by one or more wires (configuration not shown). For some applications, housing 18 comprises circuitry 26, as shown in FIG. 1B, while for other applications, the circuitry is configured to remain outside of the mouth, in which case the circuitry is typically coupled to the electrodes by one or more wires (configuration not shown).

Although device 10 is generally useful for delivery of drug molecules of all sizes, the device may be particularly useful for delivery large drug molecules (for example, biologics) that otherwise would not readily cross the sublingual membranes. In addition, device 10 may be used to deliver drugs that are injectable, or drugs for which it is desired to avoid a first pass through the liver that would reduce the bioavailability of the drug. For some applications, the drug has a solid form.

Circuitry 26 is configured to drive a low voltage radiofrequency (RF) current between the electrodes. The RF current: (a) generally dilates blood vessels in the sublingual region, thereby increasing blood flow, (b) increases pore size in the blood vessels, and/or (c) dilates junctions between epithelial cells. As a result, the RF current enhances absorption of the drug into the systemic blood circulation.

In an in vivo experiment performed on behalf of the inventor, two electrodes were applied to an outer surface of a small intestine of a rat. The electrodes were spaced 7 mm from one another. A 100 kHz, 7 V current was driven between the electrodes for between 2 and 3 seconds. Visual analysis of the tissue showed a substantial increase in dilation (and, correspondingly, blood flow) in blood vessels of the intestinal tissue after stimulation compared to prior to stimulation. The observed dilation continued for approximately 20 minutes after the conclusion of stimulation. It was also observed that tissue damage occurred if the stimulation was applied for substantially more than a few seconds. The inventor believes that RF current application similar to that applied in this experiment has a similar effect on blood vessel dilation in sublingual tissue and other non-intestinal tissue.

Typically, the RF current has a voltage of between about 4 and about 40 V, e.g., between about 4 and about 20 V. Such low voltages are effective in part because the sublingual mucosa lacks the stratum corneum that skin includes. For some applications, the RF current has a frequency of between about 30 and about 300 kHz, such as between about 80 and about 120 kHz, e.g., 100 kHz. For some applications, the circuitry applies a low frequency signal, e.g., having a frequency of between about 3 and about 40 Hz, using amplitude modulation, to a high frequency carrier signal having a frequency of between about 30 and about 300 kHz.

For some applications, circuitry 26 is configured to apply the RF current intermittently, e.g., in a series of pulses. For some applications, the RF current is applied for a period having a total duration (including non-application periods between pulses) of between one second and one minute, or between one minute and ten minutes, for causing RF dilation or ablation, and typically for a longer period of time for causing iontophoresis. The effect of the stimulation on the submucosal tissue (e.g., vasodilation) typically continues at least several minutes after the conclusion of stimulation, so that drug delivery continues after the conclusion of stimulation.

For some applications, device 10 further comprises a chemical enhancer 30 that increases permeability of the mucosa, as is known in the drug delivery art. By way of example and not limitation, chemical enhancers may be used that are described in the above-mentioned US Patent Application Publication 2008/0299079 to Meezan et al. For some applications, the chemical enhancer chemically irritates the mucosa to enhance the permeability. For example, chemical enhancer 30 may be provided as a layer of the device, such as an outermost layer that faces the floor of the mouth when device 10 is placed under the tongue, as shown in FIGS. 1A and 1B. Alternatively, the enhancer may be provided in separate regions of a layer (e.g., as points), rather than as an entire layer. The regions (e.g., points) may or may not be aligned with electrodes 22. The use of such regions may reduce the likelihood of the enhancer causing global irritation of the floor of the mouth.

For some applications, device 10 further comprises a layer of L-arginine, which causes blood vessel dilation. Alternatively, device 10 comprises L-arginine mixed with drug 20.

For some applications, device 10 further comprises a dissolvable enteric coating 32. For example, the enteric coating may be provided as a layer of the device, such as a layer between drug 20 and chemical enhancer 30, as shown in FIGS. 1A and 1B.

For some applications, device 10 further comprises an anesthetic, such as lidocaine, which typically coats the outermost layer of the housing. The anesthetic reduces (e.g., eliminates) any pain or discomfort that might be caused by the current application or chemical enhancer. Alternatively or additionally, the device may comprise a taste masking component, as is known in the art. For some applications, device 10 is configured to apply the RF current during an initial first period at an initial, relatively low first strength, which is sufficient to cause a low level of opening of junctions, which facilitates passage of some of the anesthetic. After an optional delay sufficient to allow the anesthetic to take effect (e.g., having a duration of between about 3 and about 15 minutes, e.g., about 10 minutes), during a second period device 10 applies the current at a second, greater strength to enhance passage of drug 20.

In an embodiment of the present invention, device 10 comprises the following layers (listed from innermost to outermost): a layer or core of drug 20, a first layer of chemical enhancer 30, a layer of anesthetic (e.g., lidocaine), and a second layer of enhancer 30. The second layer of enhancer 30 enhances passage of the anesthetic through the tissue generally without first causing pain. The second layer of enhancer 30 may only partially over the anesthetic, e.g., the second layer may comprise dots. Alternatively or additionally, the second layer may be relatively thin, and/or may comprise an enhancer that is weaker than that of the first layer. For applications in which the anesthetic comprises lidocaine, only relatively mild enhancement is necessary to sufficiently increase the permeability of tissue, because lidocaine is a small molecule. Relatively stronger chemical and/or electrical enhancement is then used to allow passage of the larger molecules of drug 20.

For some applications, device 10 comprises a microvibrator. During application of the current, circuitry 26 activates the microvibrator to help mask any pain or discomfort that might be caused by the current application or chemical enhancer.

For some applications, circuitry 26 automatically activates the device to apply electrical stimulation upon placement of the housing in the mouth. For example, the device may be activated by contact with liquid (e.g., saliva), such as by contact of electrodes 22 with the liquid. Optionally, such contact only occurs after one or more of the layers of the device dissolve, such as the enhancer layer or the enteric coating layer. For some applications in which device 10 comprises an anesthetic, upon sensing contact of electrodes 22 with the liquid, circuitry 26 drives the electrodes to apply the current only after a delay, e.g., having a duration of between about 2 and about 10 minutes. The delay provides time for the anesthetic to take effect before the current is applied, regardless of the properties of any coatings applied to the housing.

Alternatively, device 10 may comprise a user-controlled switch that activates application of the current. For example, the device may be placed in the mouth for drug delivery for between about five minutes and about one hour.

In an embodiment of the present invention, device 10 comprises an anesthetic (e.g., lidocaine) layer and an enhancer layer, and circuitry 26 is configured to automatically activate electrodes 22 when liquid (e.g., saliva) comes in contact with the electrodes. The circuitry drives the electrodes to apply the RF current described herein, or a typically longer-duration ablating current.

In an embodiment of the present invention, device 10 is configured to enhance delivery of drug 20 by iontophoresis, typically, but not necessarily, in combination with the RF current drug delivery enhancement techniques described herein. The device comprises two or more iontophoresis electrodes, and circuitry 26 is configured to drive a DC current between the electrodes, typically using current parameters known in the iontophoresis art. For some applications, both iontophoresis electrodes are positioned on the a layer of device 10 that comprises (e.g., is coated with) drug 20 in a vicinity of one of the electrodes (which of the electrodes depends on the polarity of the drug, as is known in the iontophoresis art). Alternatively, at least one of the electrodes is positioned on the layer of the device that comprises (e.g., is coated with) the drug, and at least another one of the electrodes is positioned on an upper surface of the device that faces the tongue when the device is placed under the tongue. In this case, the circuitry drives the current from the drug-layer electrode to the lingual electrode. For some applications, the same electrodes are used for both iontophoresis and RF current delivery, while for other applications, separate respective sets of electrodes are provided.

In an embodiment of the present invention, device 10 is configured to apply RF ablation in order to enhance drug transport across the mucous membrane by forming small openings in the membrane. Alternatively or additionally, device 10 comprises a plurality of microheaters that ablate the mucous membrane to form small openings. For example, techniques may be used for forming openings that are described in the above-mentioned U.S. Pat. No. 6,148,232 to Avrahami, or U.S. Pat. No. 6,692,456 to Eppstein et al. Further alternatively or additionally, device 10 comprises a plurality of microneedles that are configured to create small openings through the mucous membrane to enhance drug transport. As appropriate, embodiments including RF ablation, microheaters, and microneedles may be practiced using vibration or another pain-masking technique, applied by the device, during use of the device.

In an embodiment of the present invention, housing 18 is configured to have a natural springiness that applies force against the floor of the mouth when the housing is held in place by the tongue. The force helps hold the electrodes in good contact with the floor of the mouth.

A “drug,” as used in the present application, including in the claims, is to be understood broadly to include any substance that is delivered to a living organism in order to produce a beneficial effect for the treatment, cure, prevention, or diagnosis of a medical condition. For example, the drug may comprise insulin.

In an embodiment of the present invention, device 10 is used to delivery an antidote to chemical or biological warfare. The device can easily and quickly inserted to the subject's mouth by the subject, or by another person, such as a medical rescue worker or, during warfare, another soldier.

In an embodiment of the present invention, techniques described herein are implemented in a swallowable drug-delivery unit, such as a pill-shaped capsule. The unit may be configured to begin application of the RF current or other electrical stimulation when the unit reaches the small intestine, in order to increase drug uptake into the small intestine by dilating blood vessels and/or other tissues of the small intestine.

In an embodiment of the present invention, device 10 is coupled to an oral appliance, such as a bruxism appliance (e.g., a mouth guard). The device is configured to deliver the drug in front of and/or behind the teeth.

For some applications, device 10 as described with respect to any of the embodiments herein comprises an oral housing that is not a sublingual housing. The oral housing may be configured, for example, to be placed in contact with buccal mucosa, rather than under the tongue. Alternatively or additionally, the oral housing of the device is configured to be placed in contact with the front gums. For example, the housing may be horseshoe shaped, for placing around the teeth. Further alternatively or additionally, device 10 is configured for applying its current to an artery supplying the penis, in order to treat erectile dysfunction, or to a carotid artery or other brain-supplying artery, to treat stroke, or to another artery, to enhance perfusion of tissue downstream of the artery.

In an embodiment of the present invention, device 10 comprises a bioadhesive and removable liner covering the bioadhesive. The subject removes the liner to expose the bioadhesive, and places device 10 in the mouth, such that the bioadhesive adheres the device to the sublingual or buccal area.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
an oral housing, which is configured to be placed at least partially in a mouth of a subject, and which comprises a drug, and two or more electrodes;
a power supply; and
circuitry coupled to the power supply and the electrodes, and configured to drive the electrodes to drive, into oral tissue of the subject, a current having a frequency of between 30 and 300 kHz.

2. The apparatus according to claim 1, wherein the circuitry is configured to drive the electrodes to drive the current as an alternating current between the electrodes.

3. The apparatus according to claim 1, wherein the oral housing comprises a sublingual housing, which is configured to be placed at least partially under a tongue of the subject.

4. The apparatus according to claim 3, wherein the sublingual housing is U-shaped.

5. The apparatus according to claim 1, wherein the oral housing has a total volume of between 2 and 8 cm3.

6. The apparatus according to claim 1, wherein the oral housing comprises L-arginine.

7. The apparatus according to claim 1, wherein the oral housing comprises the circuitry.

8. The apparatus according to claim 1, wherein the oral housing comprises the power supply.

9. The apparatus according to claim 1, wherein the oral housing is configured to position the electrodes such that the circuitry drives the current into buccal tissue of the subject.

10. The apparatus according to claim 1, wherein the oral housing is configured to position the electrodes such that the circuitry drives the current into lip tissue of the subject.

11. The apparatus according to claim 1, wherein the circuitry is configured to drive the electrodes to drive the current as an alternating current serving as a carrier signal, and to apply a lower frequency signal to the carrier signal using amplitude modulation.

12. The apparatus according to claim 11, wherein the lower frequency signal has a frequency of between 3 and 40 Hz.

13. The apparatus according to claim 1, wherein the oral housing comprises an anesthetic.

14. The apparatus according to claim 13, wherein the circuitry is configured to:
during a first period, drive the electrodes to drive the current at a first strength sufficient to facilitate passage of some of the anesthetic into the oral tissue, and
during a second period that commences after a conclusion of the first period, drive the electrodes to drive the current at a second strength that is greater than the first strength, and sufficient to enhance passage of the drug into the oral tissue.

15. The apparatus according to claim 13, wherein the anesthetic is applied to the oral housing as a layer, and wherein the oral housing further comprises a chemical enhancer that at least partially covers the layer of anesthetic.

16. A method for delivering a drug to a subject, comprising:
receiving an oral drug delivery device including a drug, two or more electrodes, a power supply, and circuitry coupled to the power supply and the electrodes, and configured to drive the electrodes to drive, into oral tissue of the subject, a current having a frequency of between 30 and 300 kHz; and
placing the drug delivery device at least partially in a mouth of the subject.

17. The method according to claim 16, wherein placing comprises placing the drug delivery device at least partially under a tongue of the subject.

18. The method according to claim 16, wherein placing comprises placing the drug delivery device at least partially in the mouth such that at least some of the electrodes are positioned such that the circuitry drives the current into buccal tissue of the subject.

19. The method according to claim 16, wherein placing comprises placing the drug delivery at least partially in the mouth such that at least some of the electrodes are positioned such that the circuitry drives the current into lip tissue of the subject.

20. The method according to claim 16, wherein receiving comprises receiving the oral drug delivery device including the circuitry, the circuitry being configured to drive the electrodes to drive the current as an alternating current between the electrodes.

21. The method according to claim 16, wherein receiving comprises receiving the oral drug delivery device, the device including L-arginine.

22. The method according to claim 16, wherein receiving comprises receiving the oral drug delivery device including the circuitry, the circuitry configured to drive the electrodes to drive the current as an alternating current serving as a carrier signal, and to apply a lower frequency signal to the carrier signal using amplitude modulation.

23. The method according to claim 16, wherein receiving comprises receiving the oral drug delivery device, the device including an anesthetic.

24. The method according to claim 23, wherein receiving comprises receiving the oral drug delivery device that includes the anesthetic, the anesthetic being applied thereto as a layer, the device further including a chemical enhancer that at least partially covers the layer of anesthetic.

25. A method for delivering a drug to a subject, comprising:
placing the drug in a mouth of the subject;
placing two or more electrodes in the mouth; and
driving a current between the electrodes having a frequency of between 30 and 300 kHz.

26. The method according to claim 25, wherein placing the drug and the electrodes comprises placing the drug and the electrodes at least partially under a tongue of the subject.

27. The method according to claim 25, wherein driving comprises driving the current as an alternating current between the electrodes.

28. The method according to claim 25, wherein driving comprises driving the current as an alternating current serving as a carrier signal, and applying a lower frequency signal to the carrier signal using amplitude modulation.
